(19) United States
(12) Patent Application Publication
(10) Pub. No.: US 2009/0299232 A1
(43) Pub. Date: Dec. 3, 2009

(54) HEALTH MANAGEMENT DEVICE

(75) Inventors: Gerd Lanfermann, Aachen (DE); Richard Daniel Willmann, Siegburg (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/307,818

(22) PCT Filed: Jul. 2, 2007
(86) PCT No.: PCT/IB07/52560

§ 371 Date: Jan. 7, 2009

(30) Foreign Application Priority Data
Jul. 12, 2006 (EP) 06117023.9

(51) Int. Cl. A61B 5/11

(52) U.S. Cl. 600/595

(57) ABSTRACT

A method of analyzing a user's body or limb movement and to a health management system comprising a body or limb movement detecting means for detecting the movement of a user's body or limb(s), a movement analyzing means for analyzing O whether or not a result of the measurement carried out by the body or limb movement detecting means deviates from a pre-specif &#x3CA;ed value, a recording means for recording and temporarily storing the movement of the user's body or limb(s), wherein, if the result of the measurements carried out by said body movement detecting means exceeds a predetermined threshold, the movement recorded by the recording means is forwarded from the recording means to a storing means in order to provide summary information about the user's movements of the body or limb(s) exceeding the predetermined threshold.

The present invention relates to a system and method for rehabilitation and/or physical therapy for the treatment of neuromotor disorders, such as stroke. After a stroke, patients often suffer from disturbances in movement coordination. These disturbances are the least well understood, but often the most debilitating with respect to functional recovery following brain injury. These deficits in coordination are expressed in the form of abnormal muscle synergies and result in limited and stereotypic movement patterns that are functionally disabling. The result of these constraints in muscle synergies is for example an abnormal coupling between shoulder abduction and elbow flexion in the arm, which significantly reduces a stroke survivor's reaching space when he/she lifts up the weight of the impaired arm against gravity. Current neurotherapeutic approaches to mitigate these abnormal synergies have produced limited functional recovery. In the leg the expression of abnormal synergies results in coupling hip/knee extension with hip adduction. The result of this is a reduced ability of activating hip abductor muscles in the impaired leg during stance.

Two rehabilitation strategies that are used to combat these deficits are range of motion and isokinetic exercising. They are traditionally executed in manual therapy sessions between patient and therapist. In the rehabilitation strategy referred to as range of motion, the exercise of the patient consists in a movement of the user's weak arm until an extension is not further possible due to a lack of coordination (and not because the maximum extension has been reached). At this point the therapist takes over and continues the patient's motion to the point where the maximum extension is reached.

In the second rehabilitation strategy referred to as isokinetic exercising, the patient performs a certain movement under force. The patient's movement is allowed to have only a predefined speed. If he tries to be faster, a counterforce slows him down.

When traditional therapy is provided in a hospital or rehabilitation center, the patient is usually seen for half-hour sessions, once or twice a day. This is decreased to once or twice a week in outpatient therapy.

Current studies indicate that motor exercising for improving the coordination of the patient can be done at home as part of a tele-rehabilitation solution. Available systems use the videoconferencing approach, where the patient exercises in front of a camera at a time that is convenient for him. Such a system is for example disclosed in US 2002/0146672 A1. This system includes a device which senses the position of digits of a user's hand while the user is performing an exercise by interacting with a virtual image. A second device provides feedback to the user and measures the position of the digits of the hand while the user is performing an exercise by interacting with a virtual image. The virtual image is updated based on targets determined for the user's performance in order to provide harder or easier exercises. Accordingly, no matter how limited a user's movement is, if the user's performances falls within a determined parameter range, the user can pass the exercise trial and the difficulty level can be gradually increased.

The data of the user's performance is stored and reviewed by a therapist. Therefore, the rehabilitation system is comprised of a rehabilitation site, a data storage site and a data access site through an internet connection between the sites. The data access site includes software that allows a doctor/therapist to monitor the exercises performed by the patient in real time using a graphic image of the patient's hand, by sending the recorded videos to the doctor or physiotherapist, who reviews the exercises and gives feedback. There are a number of passive and active devices, e. g. Theraband or Reck MotoMed, that allow a user to perform such exercising at home as part of a tele-rehabilitation solution. However, there is still the question, how the exercising is reviewed by the therapist from a remote location. The video-recording of the complete exercising and its review at a later time by the therapist in the back-office is one of the easiest solutions, since it only requires standard components of a videoconferencing system.

The problem with such an approach is that the therapist still has to review the video in its full length. Therefore, the expensive 1:1 ratio between patient and therapist is not resolved, it is only decoupled in time and so still very time consuming. The review of exercising in a “fast forward”-mode of the recorder is the only solution at this point to accelerate the review process in order to analyze a patient's stadium or progress.

It is therefore an object of the present invention to provide a system and a method that allow accelerated analysis of a patient's or user's motor problems and/or of the progress of recovery.

This object is solved by a system and method according to claims 1 and 6 of this invention.

The health management system according to the present invention comprises a body or limb movement detecting means for detecting the movement of the user's body or limb, a movement analyzing means for analyzing whether or not a result of the measurement carried out by the body or limb movement detecting means deviates from a pre-specified value and a recording means for recording and temporarily storing the movement of the user's body or limbs. In order to provide summary information about the user's movement of the body or limbs, the data of the movement recorded by the recording means is forwarded from the recording means to a storing means if the result of the measurements carried out by said body movement detecting means exceeds a predetermined threshold. By only displaying the data of sequences including, for example, pathogenic movements or any other movements of interest, like movements showing the mobility of a patient, the time a therapist needs to analyze the limited coordination or the improvement of said motor diseases is minimized.

Therefore, the 1:1 patient:therapist ratio is not only decoupled in time, but also concentrated on the sequences being relevant for analysis and the decision how to proceed with the therapy.

In other words, the proposed system provides the recorded exercise sequence measure with annotations on the quality of the conducted exercise and the patient's compliance. Such data can be generated, for example, by tracking the essential body parts (e. g. arms, hands, face, legs) throughout a predetermined exercise. By comparing the trajectories of body parts to a reference motion, which is stored on a template and retrieved from a data base, the system can make a statement on how well the exercise was conducted, what kind of diseases the user has and in which stadium of rehabilitation he is at that moment in time. Alternatively, values such as range of motion, speed or jerk may be computed from the measured motion and compared to the reference values retrieved from the data base.

The measuring means according to the invention may be a camera-based computer-vision means with markers, a markerless motion tracking means using computer vision, inertial sensors, sensor garments and/or any other motion or position sensor. In one embodiment, the body or limb movement measuring means is at least one computer vision means, at least one visual marker and/or at least one inertial sensor. The markers or inertial sensors are placed on the respective limbs or body of the user to generate information on relative changes of position in space and may be used to compare the movements of the user doing an exercise, with a reference template. The calculation of the deviation can be performed by comparing the movement measured by the body or limb movement measuring means with a personalized exercise template based on criteria like quality, compliance or synchronicity.

The movement exceeding a threshold may indicate motor problems of the user or may indicate improvements of the user's motor problems or progress of the recovery, which is very important to evaluate the further steps in the future rehabilitation process.

Therefore, generally information on the complete exercise session as well as on specific exercises may be generated and presented to the therapist in order to provide summary information. The therapist may immediately access those sequences whose quality level lies above the predetermined threshold.

The interesting sequences in an exercise can be marked by displaying the whole video sequence, setting a specified threshold and selecting and marking those sequences whose annotated quality data exceeds the threshold. It is also possible to add data of the sequence preceding and/or following data of the marked sequence (for example nine to ten seconds each) before forwarding the summary data to the therapist, in order to indicate the formation of the movement and display the whole pathogenic movement of the user and its development. It is also possible to give statistics on a quality measure for the whole recorded exercise sequence and/or for particular exercising.

In one embodiment of the present invention, the system may further instruct the user either visually or auditorily to start and to stop exercises to facilitate the annotation and analysis process.

To support the tracking of the user's movements and to minimize the error rate of the recording of the movements, additional means such as IR-markers or inertial sensors are provided in the health management system according to the invention, which are placed on the respective limbs or the body of the user. These sensors provide further information on the motion process and may be used to compare the user's exercise with a reference template stored in the data base. A person skilled in the art will recognize that markerless tracking is also possible to get necessary information about the user's motions.

The video and the quality annotation are jointly transmitted to a therapist who is located either at a remote site where data transmission can be achieved over the internet or at the same place as the user. The therapist may immediately access those sequences whose quality measure is above a predetermined threshold. As a special case, setting the threshold to its minimum value results in the therapist accessing all parts or sequences in which movement of the user or patient takes place. The method of providing summary information about a user's health stadium according to the invention may include the following steps:
Recording of a user's motor exercising e.g. by means of computer vision supported by at least one visual marker and/or by at least one inertial sensor;
Retrieval of a personalized exercise template or reference values on motion parameters, stored in a data base;
Recognizing and analyzing the patient's movement by means of computer vision with visual markers and/or inertial sensors or by means of markerless tracking;
Calculation of a deviation measure that gives information about the deviation between the executed motion and the reference motion;
Annotating the video sequence with this information;
Reviewing of the video sequence by setting a threshold and selecting those clips whose annotated quality data exceed the threshold;
Giving statistics on the quality measure for the whole recorded exercise sequence and/or particular exercises;
Ordering of exercise clips, based on criteria like quality or compliance, from a remote site by using any possible data transfer means.

Additional details, features, characteristics and advantages of the object of the invention are disclosed in the sub-claims, Figures, examples and the following description of the respective Figures and examples—which in an exemplary fashion—show several preferred embodiments and examples of a health management device according to the invention.

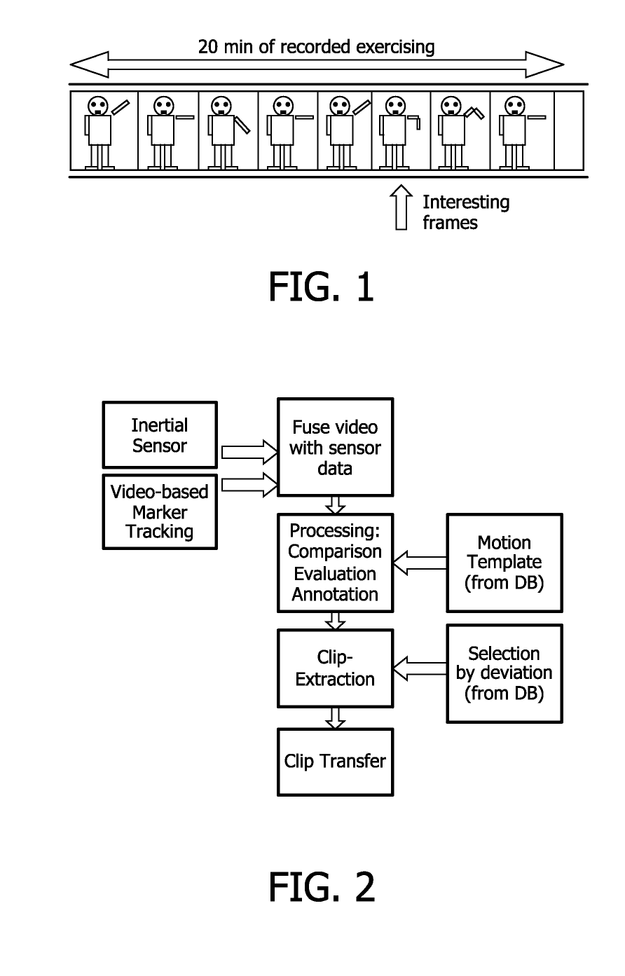

FIG. 1 shows a conventional manual review of recorded exercising;

FIG. 2 shows schematically the components of a system according to the invention;

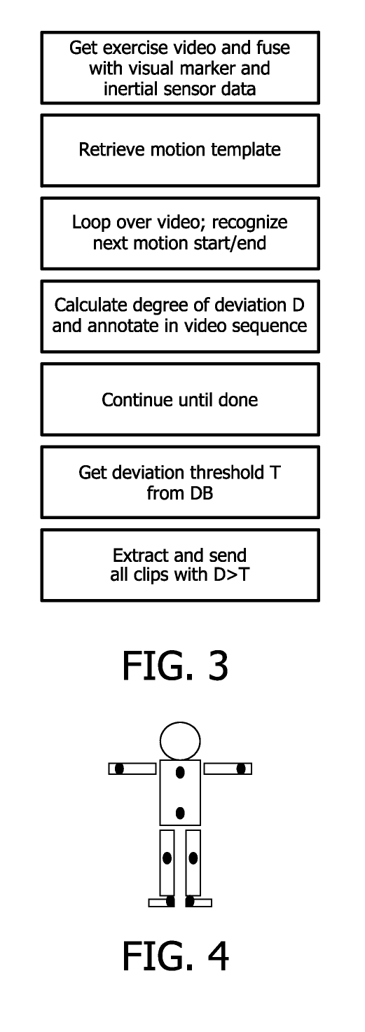

FIG. 3 shows a flow chart illustrating the method of the present invention;

FIG. 4 shows a sample placement of additional markers or inertial sensors; and

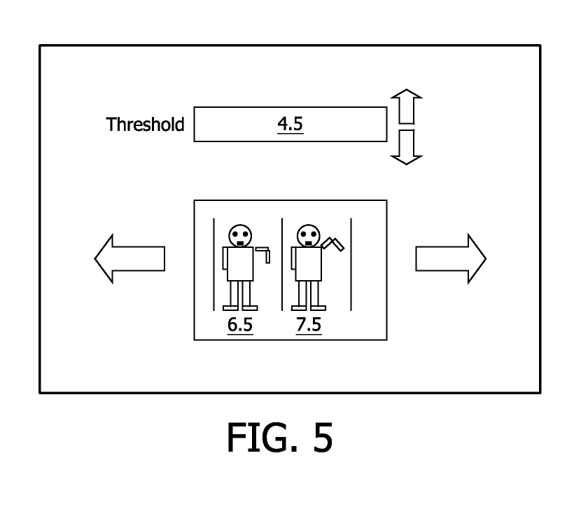

FIG. 5 shows a possible desktop of a therapist for viewing and analyzing the transmitted data.

FIG. 1 shows a conventional manual review of recorded exercising, wherein the therapist has to review the complete video although only a short sequence is relevant for the analysis, which is very time-consuming.

The health management system according to the invention is shown in FIG. 2 and a flow chart showing an embodiment of the method according to the invention is shown in FIG. 3. The health management system according to this embodiment consists of a camera system which records the user's exercises and an interaction system which instructs the user to start and to stop an exercise. The video data may be marked with these events to determine the start or the finish of the exercise at a later processing step. The video data may further be marked with an identifier of the requested exercise.

To facilitate the recording of the user's movements and to minimize the error rate of recording, the evaluation process is supported by using an additional motion tracking system comprising sensors which can be identified in the original video sequence and which are placed on the user's body or limbs as, for example, schematically shown in FIG. 4. These markers can be colored markers or IR-markers, which require the use of a corresponding IR-camera. The IR-video sequence afterwards has to be matched with the original video sequence, for example, by means of time stamps.

If inertial sensors are used (for example Magnetometer, Gyros or accelerometer) information on relative changes of the position in space of the user's limbs is generated. This data has also to be mapped to the original video sequence for example by means of time stamps.

It is not absolutely necessary to use a video sequence. Alternatively, the position in space of the user's body or limbs as measured e.g. with inertial sensors or markerless can be used to animate an avatar, the avatar's movement afterwards being reviewed by the therapist.

Afterwards, the sensor data is prepared to determine the exercise quality either by associating the position of the IR- or colored markers at a certain time with the respective video frames or by associating the information from the inertial sensors with the respective video frames.

To determine which exercise or motion template has to be retrieved from the data base, there are two possibilities. If the interaction system has visually or auditorily instructed the user to do a specific exercise, then this information is simultaneously used to retrieve the corresponding exercise or motion template from the data base. If no such information is stored and if the exercise can be assumed not to be pathological, then the video sequence may be recognized by comparing it with the relevant exercise templates in the data base.

Afterwards, the sensor and video data is used to determine the exercise quality. Therefore, the sensor data associated with video frames is used to determine the posture and motion of the patient in the respective frames (appropriate strategies for spotting motion patterns are, for example, described by Junker et al.). For this purpose, it has to be determined when exactly the different phases of an exercise occur. Compliance may be determined either by monitoring the position of the user's face with respect to the instruction screen of the user's interface or the user may be monitored for talking. High compliance is achieved if the user concentrates on the screen, low compliance is the case if the user is watching around or talking. Also early finishing of an exercise may be monitored.

If a user's section of the video material is found, in which the user has performed an exercise, a quality measure is computed. The quality measure marks the distance from the motion in the user's region to the template downloaded from a data base.

A distance can be e.g. computed using dynamic time warping as, for example, disclosed in AFU et al., Proceedings of the 31^{st} VLDB conference 2005, Trondheim 2005, which is incorporated herein by reference. Alternatively, the exercise quality may be generated from comparing target values of motion parameters such as jerk or velocity with the values of the user's or patient's movements. After evaluation and annotation of the data, the exercise quality is attached to the original video sequence.

Finally, the annotated video is transferred to the backend and stored in the data base. The video sequence is reviewed in a browser as illustrated in FIG. 4. The video sequences are reviewed by setting a threshold and selecting and marking those clips whose annotated quality data exceeds a predetermined threshold. While browsing, the system may display the following:

1. statistics on the general quality and compliance

2. statistics on the exercise groups, for example leg exercises or arm exercises, and the respective compliance.

Additionally, the function of sorting the clips according to various criteria can be provided. Criteria can be, for example, the worst exercise first, the first non-compliant exercise, the first or worst exercise first and so on. The video sequence may finally be browsed and reviewed by setting a threshold parameter. The clips exceeding the threshold are displayed. It is possible to change or adjust the summary data by changing or adjusting the threshold (see, for example, in FIG. 5 a possible desktop sketch of a program in which the function of adjustment of the threshold is provided).

1. A health management system comprising:
a body or limb movement detecting means for detecting the movement of a user's body or limb,
a movement analyzing means for analyzing whether or not a result of the measurement carried out by the body or limb movement detecting means deviates from a pre-specified value,
a recording means for recording and temporarily storing the movement of the user's body or limb, and
a storing means for storing the movement of the user's body or limb, wherein the movement recorded by: the recording means is forwarded to the storing means, if the result of the measurements carried out by said body movement detecting means exceeds a predetermined threshold.

2. The health management system according to claim 1,
wherein the body or limb movement-measuring means is at least one camera-based computer-vision means with markers or a markerless motion tracking means using computer vision and/or at least one inertial sensor, at least one sensor garments and/or any other motion or position sensor.

3. The health management system according to claim 1,
wherein the calculation of the deviation is performed by a comparison between the movement measured by the body or limb movement-measuring means and a personalized exercise template, based on criteria like quality and/or compliance and/or synchronicity.

4. The health management system according to claim 1,
wherein the movements exceeding the threshold indicate motor problems of the user.

5. The health management system according to claim 1,
wherein the movements exceeding the threshold indicate improvements of a user's motor problems.

6. The health management system according to claim 1,
wherein the threshold is tunable by the therapist in order to refine the analysis of the user's movement.

7. A method for analyzing a user's body or limb movement after a stroke or other neurological diseases, including:
recording the a user's body or limb movement while the user is doing a predetermined motor exercise,
comparing said movement with a personalized exercise template,
recognizing and analyzing the user's movements by using an analyzing means,
marking sequences in which the user's movement deviates from the template by an amount exceeding a predetermined threshold,
forwarding the marked sequences to a storing means in order to provide selected information about the user's movements of the body or limb, wherein the forwarded sequences can be retrieved by a therapist for evaluation of at least one of the user's condition and the user's progress of recovery.

8. (canceled)

9. The method of analyzing a user's body or limb movement according to claim 7, wherein the therapist is located at a remote site where data transmission can be achieved over the Internet.

10. The method of analyzing a user's body or limb movement according to claim 7, wherein data of the sequence preceding and/or following the marked sequence is added before forwarding and transmitting the summary data to the therapist, for indicating the formation of the movement.
